(19) United States
(12) Patent Application Publication
(10) Pub. No.: US 2025/0384549 A1
(43) Pub. Date: Dec. 18, 2025

(54) Method for Diagnosing Keratoconus Using Purkinje Image Geometry

(71) Applicant: Mohsen Sharifzadeh, Cottonwood Heights, UT (US)

(72) Inventor: Mohsen Sharifzadeh, Cottonwood Heights, UT (US)

(21) Appl. No.: 18/746,773

(22) Filed: Jun. 18, 2024

(51) Int. Cl. G06T 7/00; G16H 30/40; G16H 50/20

(52) U.S. Cl. CPC G06T 7/0012; G16H 30/40; G16H 50/20; G06T 2207/10101; G06T 2207/30041

(57) ABSTRACT

This invention relates to a method for diagnosing keratoconus by analyzing the geometric properties of Purkinje images. The method involves capturing Purkinje images of the cornea using a low-cost and simple device, such as a smartphone attachment, making it more accessible than current diagnostic tools like slit lamps. By comparing these images to reference images from healthy corneas, significant shifts and distortions are identified as indicators of keratoconus. This non-invasive tool provides an accessible and cost-effective means for early detection and ongoing monitoring. The method's reliability is ensured through extensive data collection from both healthy and keratoconic corneas. This approach offers primary assessments, especially in areas lacking access to advanced medical care, potentially reducing the need for more invasive procedures.

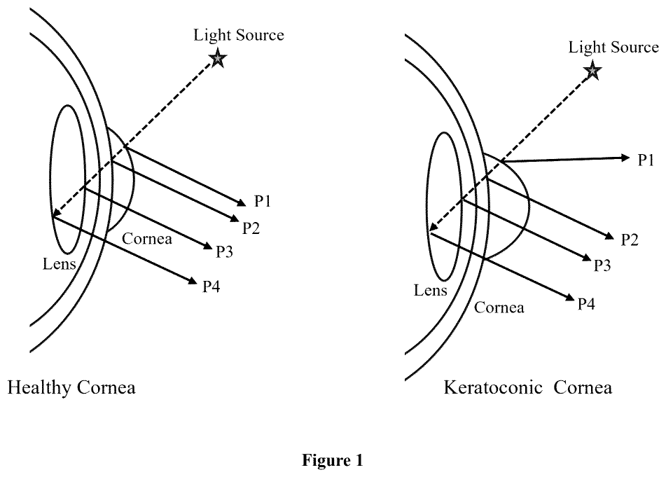

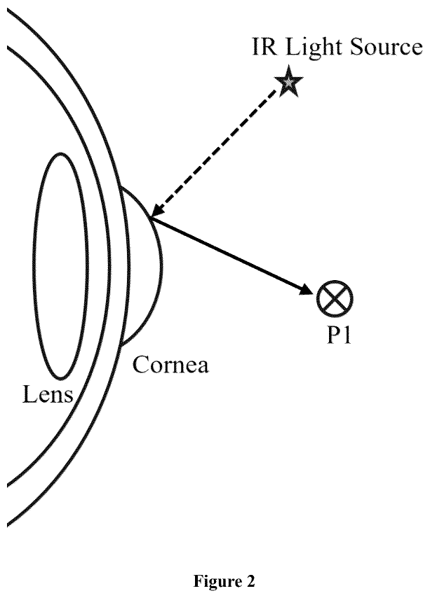

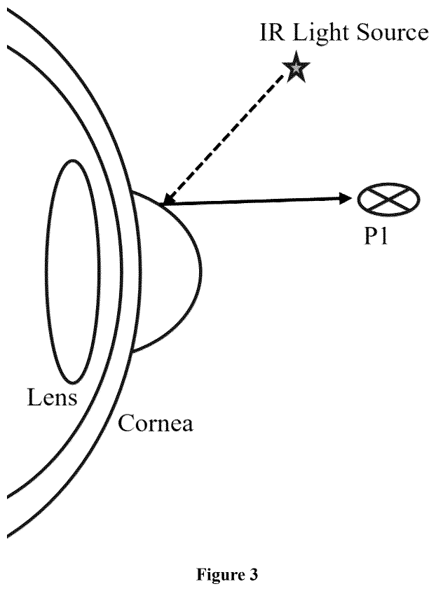

### BACKGROUND OF THE INVENTION

Keratoconus is a progressive eye disease characterized by the thinning and bulging of the cornea into a cone-like shape, leading to distorted vision and potentially severe visual impairment if left untreated. Current diagnostic methods primarily rely on corneal topography, tomography, and other sophisticated imaging techniques that may not be readily accessible in all healthcare settings.

Purkinje images, reflections from the corneal and lens surfaces, have traditionally been used for eye tracking and assessing optical properties. However, their potential for diagnosing corneal abnormalities such as keratoconus has not been fully explored. The unique geometric distortions and shifts in Purkinje images caused by the irregular corneal surface in keratoconus present an opportunity for a novel diagnostic approach.

This invention proposes a method to analyze the first Purkinje image, primarily affected by keratoconus, to detect these geometric changes. By providing a non-invasive, accessible diagnostic tool, this method aims to facilitate early detection and monitoring of keratoconus, improving patient outcomes and reducing the burden on healthcare systems.

Additionally, this method's simplicity allows for the use of low-cost devices, including smartphone attachments, to capture and analyze Purkinje images. This accessibility can be particularly beneficial in regions with limited access to advanced diagnostic tools, providing primary assessments and early indications of keratoconus.

### SUMMARY OF THE INVENTION

The present invention relates to a novel method for diagnosing keratoconus by analyzing the geometric properties of Purkinje images, particularly focusing on the displacement and distortion of these images. This method provides an accessible, non-invasive, and cost-effective means for early detection and ongoing monitoring of keratoconus, leveraging low-cost devices such as smartphone attachments.

The method involves capturing Purkinje images of the cornea using a simple optical setup. The captured images are processed to isolate the Purkinje images, with a specific focus on the first Purkinje image (P1). Key geometric properties, including the displacement and distortion of P1, are measured and compared to baseline metrics derived from healthy corneas.

A robust algorithm is employed to ensure accurate alignment using the pupil center as a reference point. The algorithm calculates the Euclidean distance from the pupil center to P1 and quantifies the distortion by measuring the area, perimeter, and eccentricity of P1. These metrics are compared against a database of reference values to diagnose keratoconus.

Additionally, the method allows for rotating the light source to obtain multiple scores (K1 and K2) from different directions, capturing the asymmetry and extent of corneal irregularities for a more comprehensive assessment. Diagnostic criteria and a scoring system are developed to assess the severity of keratoconus based on the degree of displacement and distortion.

By providing a reliable and accessible diagnostic tool, this invention aims to facilitate early detection and monitoring of keratoconus, particularly in regions with limited access to advanced medical care, potentially reducing the need for more invasive procedures.

### DETAILED DESCRIPTION OF THE INVENTION

1. Introduction:
   This invention provides a method for diagnosing keratoconus by analyzing the geometric properties of Purkinje images. By capturing and analyzing these images, significant shifts and distortions indicative of keratoconus can be identified, offering a non-invasive and accessible diagnostic tool.
2. Capturing Purkinje Images:
   Optical Setup:
      The method involves using a simple and low-cost device, such as a smartphone attachment or a small form factor camera, to capture Purkinje images of the cornea.
      The setup includes a light source to illuminate the eye and a camera to capture the reflections. Consistent lighting and subject positioning are essential to ensure accurate and repeatable image capture.
   Procedure:
      The subject is positioned with the eye exposed to the light source. The camera captures multiple Purkinje images, primarily focusing on the first Purkinje image, which is the reflection from the outer surface of the cornea. However, the second, third, and fourth Purkinje images can also be captured to assist in geometric validation.
      Multiple images can be taken to ensure consistency.
3. Geometric Analysis of Purkinje Images:
   Image Processing:
      The captured images are processed using software that isolates and extracts the Purkinje images. Key points, such as the center and edges of each image, are identified.
   Measurement of Displacement:
      The displacement of the first Purkinje image from its expected position in a healthy cornea is measured. This involves comparing the position of the Purkinje image in the subject's eye to reference images from healthy corneas.
   Quantification of Distortion:
      The shape and size of the Purkinje images are analyzed to quantify any distortions. Metrics such as axis lengths, area, and perimeter are compared to standard metrics from healthy corneas.
   Geometric Validation Using Multiple Purkinje Images:
      The geometric relationships between the first and subsequent Purkinje images (second, third, and fourth) are analyzed to ensure the accuracy of the captured images. This involves verifying that the images are correctly aligned and correspond to the correct reflections.
   Algorithm Development:
      Algorithms are developed to automate the analysis, providing consistent and objective measurements of displacement and distortion. These algorithms use geometric and mathematical principles to detect significant changes indicative of keratoconus.
4. Geometric/Mathematical Algorithm for Diagnosing Keratoconus

The proposed method leverages the geometric properties of Purkinje images to diagnose keratoconus. The following algorithm outlines the steps required to measure the displacement and distortion of the Purkinje image (P1) while ensuring accurate alignment using the pupil center as a reference point.

### 4.1 Define Coordinate System

Establish a coordinate system with the origin (x₀, y₀) at the detected center of the pupil on the camera sensor.

### 4.2 Capture and Preprocess Image

Capture the image of the subject's cornea and preprocess it to enhance reflection points and reduce noise.

### 4.3 Detect Pupil Center

Use robust image processing techniques to detect the center of the pupil:
Apply edge detection algorithms (e.g., Canny edge detector) to identify the boundaries of the pupil.
Enhance the image contrast to distinguish the pupil from the surrounding iris.
Fit an ellipse to the detected edges to model the pupil's shape and determine the center of the ellipse as the detected pupil center.

### 4.4 Normalize Image Alignment

Align the image such that the detected pupil center matches the reference point (x₀, y₀) on the sensor.

### 4.5 Identify Reflection Points (P1)

Detect the coordinates (XP1, YP1) of the reflection point Pl on the sensor.

### 4.6 Measure Distance and Analyze Distortion

Calculate the Euclidean distance from the pupil center to P1:

$${d}_{P 1} = \sqrt{{( {x}_{P 1} - {x}_{0} )}^{2} + {( {y}_{P 1} - {y}_{0} )}^{2}}$$

Analyze the shape and properties of the reflection around P1 by measuring the area, perimeter, and eccentricity of the reflection spot:
Area (A): Measure the area of the reflection spot.
Perimeter (P): Measure the perimeter of the reflection spot.
Eccentricity (e): Calculate the eccentricity of the ellipse fitting the reflection spot:

$$e = \sqrt{1 - \frac{{b}^{2}}{{a}^{2}}}$$

where a is the semi-major axis and b is the semi-minor axis of the ellipse.

### 4.7 Establish Baseline Metrics

Develop a database of baseline metrics (distances, areas, perimeters, and eccentricities) from a large sample of healthy corneas to serve as reference values.

### 4.8 Comparison and Thresholds

Compare the measured metrics of the patient's cornea with the database metrics. Use statistical methods to determine whether the patient's measurements fall within the normal range or indicate keratoconus:
Distance Threshold: Compare d_{P1} to the average and standard deviation of distances in the healthy cornea database.

$$\vert {d}_{P 1} - {μ}_{{d}_{P 1}} \vert > k \cdot {σ}_{{d}_{P 1}}$$

Where μ_{d}_{_{P1}} is the mean distance for healthy corneas, σ_{d}_{_{P1}} is the standard deviation, and k is a chosen confidence factor (e.g., 2 for 95% confidence).
Similarly, compare other distortion metrics (area, perimeter, and eccentricity) with the reference values using their respective means and standard deviations.

### 4.9 Scoring System

Develop a scoring system that combines the deviations in each metric to provide a comprehensive assessment of the cornea's condition:

$$Score = {w}_{d} \cdot \frac{\vert {d}_{P 1} - {μ}_{{d}_{P 1}} \vert}{{σ}_{{d}_{P 1}}} + {w}_{A} \cdot \frac{\vert A - {μ}_{A} \vert}{{σ}_{A}} + {w}_{e} \cdot \frac{\vert e - {μ}_{e} \vert}{{σ}_{e}}$$

where w_{d}, w_{A}, and wₑ are weights assigned to each metric.
4.10 Rotating the Light Source: To enhance diagnostic accuracy, the light source can be rotated to obtain multiple scores (K1 and K2) from different directions. This multidirectional assessment captures the asymmetry and extent of irregularities in the corneal surface, providing a more comprehensive diagnosis.

### Mathematical Formulation

1. Transformation:

$${x}_{P 1}^{′} = {x}_{P 1} \cdot \frac{real\_width}{w} \\ {y}_{P 1}^{′} = {y}_{P 1} \cdot \frac{real\_height}{h}$$

2. Distance Calculation:

$${d}_{P 1} = \sqrt{{( {x}_{P 1}^{′} - {x}_{0} )}^{2} + {( {y}_{P 1}^{′} - {y}_{0} )}^{2}}$$

3. Threshold Comparison:

$$\vert {d}_{P 1} - {μ}_{{d}_{P 1}} \vert > k \cdot {σ}_{{d}_{P 1}}$$

4. Scoring System:

$$Score = {w}_{d} \cdot \frac{\vert {d}_{P 1} - {μ}_{{d}_{P 1}} \vert}{{σ}_{{d}_{P 1}}} + {w}_{A} \cdot \frac{\vert A - {μ}_{A} \vert}{{σ}_{A}} + {w}_{e} \cdot \frac{\vert e - {μ}_{e} \vert}{{σ}_{e}}$$

5. Diagnostic Criteria:
   Thresholds for Diagnosis:
      Diagnostic criteria are established based on the measured geometric properties. Thresholds for displacement and distortion are defined, indicating the presence of keratoconus.
   Scoring System:
      A scoring system is developed to assess the severity of keratoconus based on the degree of displacement and distortion. This system provides a quantitative measure of the condition, aiding in diagnosis and monitoring.
6. Validation and Comparison:
   Clinical Studies:
      Clinical studies are conducted to validate the method against existing diagnostic techniques, such as corneal topography and tomography. The accuracy and reliability of the method are evaluated through these studies.
   Comparative Analysis:
      The results of the Purkinje image analysis are compared to traditional diagnostic methods to ensure consistency and reliability. This comparison demonstrates the method's efficacy in diagnosing keratoconus.
7. Applications and Benefits:
   Accessibility:
      The simplicity and low cost of the method make it accessible to a wide range of healthcare settings, including regions with limited access to advanced diagnostic tools.
   Early Detection:
      By providing a means for early detection, the method can help prevent severe visual impairment and reduce the need for invasive treatments.
   Non-Invasive Monitoring:
      The method offers a non-invasive way to monitor the progression of keratoconus, allowing for timely intervention and better management of the condition.

1. A method for diagnosing keratoconus comprising the steps of capturing Purkinje images of a subject's cornea using a low-cost device, analyzing both the displacement and distortion of said Purkinje images, and comparing the analyzed geometric properties to predetermined criteria indicative of keratoconus, comprising the steps of:
Capturing Purkinje images of a subject's cornea using a low-cost device.
Analyzing the geometric properties of said Purkinje images.
Comparing the analyzed geometric properties to predetermined criteria indicative of keratoconus.

2. The method of claim 1, wherein the geometric properties analyzed include the position, shape, and size of the first Purkinje image.

3. The method of claim 1, wherein the analysis involves measuring the displacement of the first Purkinje image from its expected position in a healthy cornea.

4. The method of claim 1, wherein the analysis involves quantifying the distortion of the first Purkinje image by comparing it to standard metrics from healthy corneas.

5. The method of claim 1, further comprising capturing additional Purkinje images (second, third, and fourth) to assist in geometric validation.

6. The method of claim 5, wherein the geometric relationships between the first Purkinje image and the subsequent Purkinje images are analyzed to ensure the accuracy of the captured images.

7. The method of claim 1, wherein algorithms are developed to automate the analysis, providing consistent and objective measurements of displacement and distortion.

8. The method of claim 1, wherein diagnostic criteria are established based on the measured geometric properties, with thresholds for displacement and distortion defined to indicate the presence of keratoconus.

9. The method of claim 8, further comprising developing a scoring system to assess the severity of keratoconus based on the degree of displacement and distortion.

10. The method of claim 1, wherein the device used for capturing Purkinje images is a smartphone attachment.

11. The method of claim 1, wherein the method is validated through clinical studies comparing the results to existing diagnostic techniques such as corneal topography and tomography.

12. The method of claim 1, wherein the method provides primary assessments and early indications of keratoconus, particularly in regions lacking access to advanced medical care.

13. The method of claim 1, wherein the Purkinje images are captured using a light source, including but not limited to infrared (IR) light.

14. The method of claim 1, further comprising using a geometric algorithm to measure the displacement and distortion of the Purkinje image (P1) and comparing these measurements to a database of reference values to diagnose keratoconus.

15. The method of claim 14, wherein the geometric algorithm includes steps for capturing the Purkinje image, identifying the pupil center and reflection points, normalizing alignment, calculating distances, and quantifying distortion metrics such as area, perimeter, and eccentricity.

16. The method of claim 14, wherein the comparison involves using statistical methods to determine whether the measured metrics fall within the normal range of the reference database.

17. The method of claim 1, further comprising rotating the light source to obtain multiple scores (K1 and K2) from different directions to enhance the diagnostic accuracy of keratoconus.
This detailed algorithm and associated claims ensure that the diagnostic method is clearly defined and demonstrates the technical innovation and practical application of the method for diagnosing keratoconus using Purkinje images.
